(19) United States
(12) Patent Application Publication
(10) Pub. No.: US 2010/0331602 A1
(43) Pub. Date: Dec. 30, 2010

(54) FOCUSED MAGNETIC FIELDS

(76) Inventors: David J. Mishelevich, San Mateo, CA (US); M. Bret Schneider, San Mateo, CA (US)

Correspondence Address: SHAY GLENN LLP, 2755 CAMPUS DRIVE, SUITE 210, SAN MATEO, CA, 94403, US

(21) Appl. No.: 12/677,220

(22) PCT Filed: Sep. 9, 2008
(86) PCT No.: PCT/US08/75706

§ 371 Date: Sep. 16, 2010

Related U.S. Application Data
(60) Provisional application No. 60/970,964, filed on Sep. 9, 2007.

(51) Int. Cl. A61N 2/02

(52) U.S. Cl. 600/13

(57) ABSTRACT

Devices, systems and methods are provided applicable to Transcranial Magnetic Stimulation (TMS) for focusing the magnetic fields generated by electromagnets. In particular, devices, systems and methods including focusing electromagnets and focusing shapes are described.

### CROSS REFERENCE TO RELATED APPLICATIONS

This application claims priority to U.S. Provisional Patent Application Ser. No. 60/970,964, filed on Sep. 9, 2007, titled “FOCUSED MAGNETIC FIELDS.” This application is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The devices and methods described herein relate generally to the focusing of magnetic fields generated by electromagnets used for Transcranial Magnetic Stimulation.

### BACKGROUND OF THE INVENTION

Transcranial Magnetic Stimulation (TMS) typically involves the application of electromagnetic fields to one or more target brain regions in order to excite or inhibit the target brain regions. For example, a single or double standard TMS coil placed on a patient's scalp and operated at a power level at, or slightly above, a patient's motor threshold will directly active neurons from the cortical crowns to the bottom of the cortical gyri- a depth of about 1-3 cm. Using this approach, deeper structures (herein referred to as “sub-cortical”, even when these deeper areas are histologically layered in nature) are activated only secondarily through intracerebral neural connections. Conventional approaches typically do not reach greater depths (for example, the cingulate gyms, the insula and other sub-cortical structures). Deep brain modulation cannot be accomplished by simply turning up the power of the stimulating electromagnet, because the intervening tissue, for example superficial cortex, will be over-stimulated, causing undesired side effects such as seizures.

Furthermore, the size of the precision with which a target brain region may be stimulated is related to the ability of the TMS system to focus on only the target brain region or fibers feeding into the target brain region, but not non-target regions. This is true both in deep brain regions as well as more superficial (e.g., cortical) targets.

For example, positive outcomes for treatment of depression refractory to drug treatment have been demonstrated with repetitive Transcranial Magnetic Stimulation (rTMS, Avery et al., 2005). rTMS works indirectly, because the superficial stimulation of the dorsolateral pre-frontal cortex is carried by nerve fibers to the deeper cingulate gyrus. More effective therapy of depression and treatment of a number of other conditions such as chronic pain, addiction, obesity, and obsessive compulsive disorder would be possible with improved focused brain stimulation, including focused stimulation capable of reaching depths below the cortex. Devices for providing deep brain stimulation with Transcranial Magnetic Stimulation are described in Schneider and Mishelevich, U.S. patent application Ser. No. 10/821,807 and Mishelevich and Schneider, U.S. patent application Ser. No. 11/429,504. Whether superficial or deep stimulation is being employed, focusing the applied magnetic field during TMS has the potential to improve clinical results. In particular, the ability to stimulate at depth could be facilitated by shaping the profile of the magnetic field of one or more primary stimulating electromagnets, thereby focusing their magnetic fields and more preferentially stimulating a given targeted neural structure.

The magnetic fields used for Transcranial Magnetic Stimulation determine both the depth and size of the region of stimulation. Thus, a more focused magnetic field would be capable of stimulating an area that is also more tightly focused, and may be better controlled by the TMS system.

TMS electromagnets comprising coils having non-flat configurations (for example “butterfly” double coils) are known, and are commercially available from vendors including by Magstim LTD (Wales, UK) and the Dantec (a subsidiary of Medtronic, Copenhagen, Denmark). While some of these configurations may be useful (with modification) for focusing the electromagnetic filed to exclude lateral field from the patient's head, in many cases the central portion of the field is weakened by the angular separation of the central coil wire elements making such coils weak when used in the reverse-faced position.

Described herein are systems, methods and devices for improving the focus of the primary electromagnet(s) used for Transcranial Magnetic Stimulation, which may allow for enhanced stimulation of targeted neural structures, and may address the problems described above.

### SUMMARY OF THE INVENTION

Described herein are devices and systems configured to enhance the focus of the emitted magnetic field of a primary TMS electromagnets so the shape/width of the field is narrower than currently used (e.g., flat or butterfly) TMS electromagnets. Two complementary solutions for focusing the magnetic field emitting by a primary Transcranial Magnetic Stimulation electromagnet are described herein, attaching one or more focusing electromagnets to the TMS electromagnet and configuring the TMS electromagnet into a focusing shape. These approaches maybe used separately or in combination.

In some variations, one or a plurality of smaller focusing electromagnets are placed at appropriate locations behind the primary TMS electromagnet. The electromagnetic field of the primary electromagnet is modified (e.g., counterbalanced) by the fields of the focusing electromagnets and thus is narrowed.

In some variations, primary TMS electromagnet is formed or shaped into a focusing shape so that the center of the double coil presents a substantially planar face that is configured to be placed closer to the target region than the lateral margins of the TMS electromagnet. For example, a standard double coil TMS electromagnet may be re-configured so that the center of the double coil is positioned physically closer to the brain than to the lateral margins of the coils, but retaining a substantially planar configuration in the center portion where the two coils pass currents in the same direction. This coil configuration maximizes delivery of energy to the target from the central planar portion of the coil.

For example, described herein are Transcranial Magnetic Stimulation system for stimulating a neuronal target, the system comprising: at least one primary Transcranial Magnetic Stimulation electromagnet; a focusing electromagnet adjacent to the primary Transcranial Magnetic Stimulation electromagnet; and a stimulation controller configured to coordinate activation of the primary Transcranial Magnetic Stimulation electromagnet and the focusing electromagnet, so that the field emitted by the focusing electromagnet focuses the magnetic field emitted by the primary electromagnet. The system may also include a frame or gantry to which the primary TMS electromagnet(s) may be attached or otherwise supported.

In some variations, the system includes a plurality of focusing electromagnets adjacent to the primary Transcranial Magnetic Stimulation electromagnet. Similarly, the system may include a plurality of Transcranial Magnetic Stimulation electromagnets, wherein each of the Transcranial Magnetic Stimulation electromagnets is focused by one or more adjacent focusing electromagnets.

In some variations, the system includes a distributor communicating with the stimulation controller. The distributor may synchronize activation of the primary TMS electromagnet and the focusing electromagnet(s) that modify the field of the primary TMS electromagnet. In general, both the primary TMS electromagnet and the focusing electromagnets are powered (so that they emit fields) at the same time.

In some variations, the system includes a separate Transcranial Magnetic Stimulation power source and a focusing electromagnet power source.

Also described herein are Transcranial Magnetic Stimulation systems for stimulating a neuronal target that include: at least one primary Transcranial Magnetic Stimulation electromagnet having a focusing shape. The focusing shape of the primary Transcranial Magnetic Stimulation electromagnet comprises a planar region (corresponding to the area of maximal field intensity emitted by the primary Transcranial Magnetic Stimulation electromagnet), wherein the planar region is configured to be placed in the closest location to the intended target. In the focusing shape, the more lateral region of the primary TMS electrode are directed backwards and away from the planar region, whereby the magnetic field produced by lateral regions is separated from the neuronal target region. In general, primary TMS electromagnets configured in a focusing shape are formed from two (or more) coils, such as the “figure-eight” shaped TMS electromagnets described herein. In some variations, these shapes may be formed of a double coil magnet that has been bent so that the region of contact between the two coils (the region of intersection) includes the planar region, and the more lateral regions outside of this region of contact are bent away from the lateral face. This TMS electromagnet is configured so that the lateral face is positioned closest to the neuronal target.

Thus, in general, the primary Transcranial Magnet Stimulation electromagnet having a focusing shape may comprise a plurality of joined coils, wherein the planar region is formed by the intersection of the coils, further wherein the coils are configured so that the current running through the coils in the intersection region travels in essentially the same direction for all of the coils.

Also described herein are Transcranial Magnetic Stimulation systems having both focusing shapes and focusing electromagnets. For example, described herein are TMS systems for stimulating a neuronal target comprising: at least one primary Transcranial Magnetic Stimulation electromagnet having a focusing shape; a focusing electromagnet adjacent to the primary Transcranial Magnetic Stimulation electromagnet; and a stimulation controller configured to coordinate activation of the primary Transcranial Magnetic Stimulation electromagnet and the focusing electromagnet so that the field emitted by the focusing electromagnet narrows and focuses the magnetic field emitted by the primary electromagnet.

Also described herein are methods of using the focused primary TMS electromagnets described herein. For example, described herein are Transcranial Magnetic Stimulation methods for stimulating a neuronal target tissue, including the steps of: positioning at least one primary Transcranial Magnetic Stimulation electromagnet adjacent to a subject's head; and emitting a magnetic field from the primary Transcranial Magnetic Stimulation electromagnet toward a neuronal target, and focusing the field emitted by the Transcranial Magnetic Stimulation electromagnet by energizing a focusing electromagnet located adjacent to the primary Transcranial Magnetic Stimulation electromagnet, so that the field emitted by the focusing electromagnet counterbalances a portion of the magnetic field emitted by the primary electromagnet.

The step of emitting a focused magnetic field may include the step of energizing a plurality of focusing electromagnets located adjacent to the primary Transcranial Magnetic Stimulation electromagnet.

In some variations, the step of emitting a focused magnetic field comprises controlling a distributor to synchronize the power supplied to the primary Transcranial Magnetic Stimulation Electromagnet and the focusing electromagnet so that they fire simultaneously.

These methods may also include the step of aiming the primary Transcranial magnetic Stimulation electromagnet at a neuronal target.

Also described herein are Transcranial Magnetic Stimulation methods for stimulating a neuronal target tissue using a TMS electromagnet having a focusing shape. For example, a TMS method may include the steps of: positioning at least one primary Transcranial Magnetic Stimulation electromagnet adjacent to a subject's head, wherein the primary Transcranial Magnetic Stimulation is configured in a focusing shape having a planar region corresponding to the area of maximal field intensity emitted by the primary Transcranial Magnetic Stimulation electromagnet and lateral regions extending backwards and away from the planar region; orienting the primary Transcranial Magnetic Stimulation electromagnet so that the planar region is positioned toward the neuronal target; and emitting a magnetic field from the primary Transcranial Magnetic Stimulation electromagnet toward the neuronal target.

### BRIEF DESCRIPTION OF THE DRAWINGS

FIG. 1A shows a prior art double-coil electromagnet.

FIG. 1B illustrates the three-dimensional magnetic field profile for the double-coil electromagnet shown in FIG. 1A.

FIG. 2A shows one variation of a TMS electromagnet including attached field-modifying electromagnets (focusing electromagnets) connected thereto.

FIG. 2B illustrates the three-dimensional magnetic field profile for the combination of the double-coil electromagnet and associated focusing electromagnets shown in FIG. 2A.

FIG. 3 illustrates a schematic diagram of one variation of a TMS system including power distribution and control to support a TMS electromagnet and its focusing electromagnets.

FIGS. 4A-4D illustrate variations of TMS electromagnets configured to enhance and focus the magnetic field, as described herein.

FIG. 4E illustrates a partial cross-section through one variation of a TMS electromagnet similar to the TMS electromagnet shown in FIG. 4B.

### DETAILED DESCRIPTION OF THE INVENTION

The methods, devices and systems for focusing and/or otherwise modifying the magnetic field of a Transcranial Magnetic Stimulation electromagnet described herein are appropriate for use with any appropriate variation of Transcranial Magnetic Stimulation (TMS), including repetitive Transcranial Magnetic Stimulation (rTMS). In particular, described herein are devices, systems, and methods that may include either primary TMS electromagnet(s) having a focusing shape, and/or including one or more focusing electromagnets.

In general, these devices, systems and methods may focus the emitted magnetic field of a primary electromagnet used for TMS (e.g., “a primary TMS electromagnet”). In any of the systems described herein multiple primary TMS electromagnets may be used to apply electromagnetic energy during TMS.

As used herein the term “focus” or “focusing” when referring to electromagnetic field refers to shaping and/or narrowing the field. The fall off of the field near the periphery may be increased, so that the emitted field is narrower. Ideally the peak strength of the emitted filed is not substantially reduced, particularly within the region of greatest field strength, although the peripheral region may be reduced.

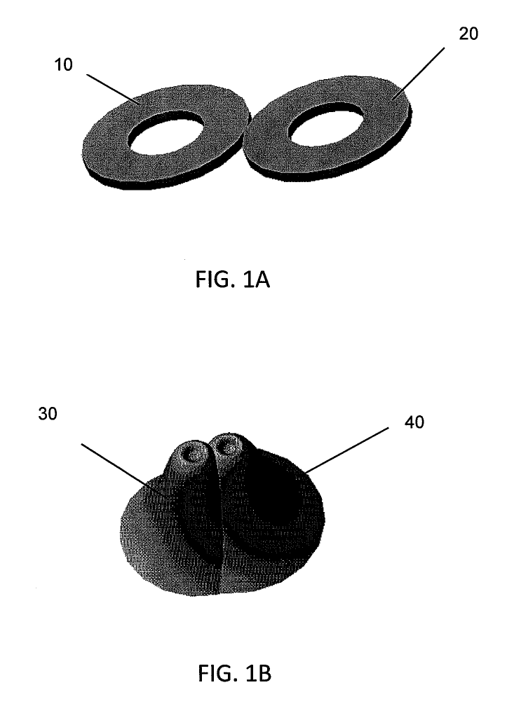

FIG. 1A shows one variation of a ‘standard’ (e.g., prior art) primary TMS electromagnet. This variation is configured as a typical double figure-eight electromagnet with component coils 10 and 20. As shown on FIG. 1B, the magnetic fields produced by the two coils 10 and 20 overlap with hump 30 produced by coil 10 and hump 40 results from stimulation by coil 20. Such figure-eight double coils are well known, for instance the 70 mm double-coil configuration from Magstim (Model 9925, Wales, UK).

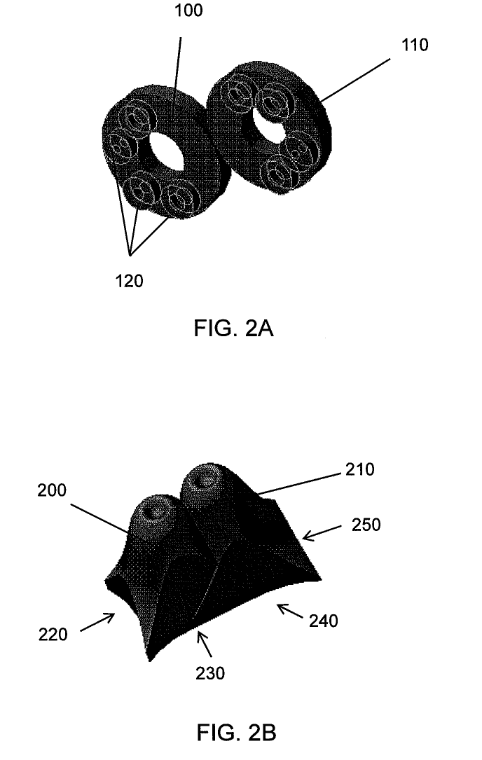

The magnetic field profile produced by this standard TMS electromagnet may be focused or otherwise desirably modified by the use of either (or both) focusing electromagnets or a focusing shape. For example, FIG. 2A illustrates a primary TMS electromagnet similar to the one shown in FIG. 1A including a plurality of focusing electromagnets. In FIG. 2A, eight focusing electromagnets are shown attached to the back side of the primary electromagnet. The primary electromagnet is a dual electromagnet including a figure-eight double coil (coils 100 and 110), to which eight smaller magnets 120 have been attached.

In this variation, the eight focusing electromagnets are secured immediately adjacent (e.g., in contact with) the back of the primary TMS electromagnet. In this context, the “back” of the TMS electromagnet is the side of the TMS electromagnet that will face away from the target site within the patient. In some variation, one or more focusing electromagnets may be placed adjacent to the sides of the primary TMS electromagnet, rather than behind it. In some variations, the focusing electromagnets are separated from the primary TMS electromagnet by a space that may include one or more materials, particularly materials that are magnetically permeable. In FIG. 2A, the electromagnets are secured to the TMS electromagnet.

In this example eight focusing electromagnets are shown. Fewer or greater numbers of focusing electromagnets may be used. It may be advantageous to provide a plurality of focusing electromagnets, in order to more precisely shape, and focus, the field emitted by the primary TMS electromagnet. In variations in which a plurality of focusing electromagnets are used, focusing electromagnets having different shapes, and/or strengths may be used at different positions adjacent to the primary electromagnet in order to focus or shape the emitted field of the primary TMS electromagnet. For example, in FIG. 2A focusing electromagnets having approximately equivalent strengths are placed in complementary (e.g., symmetric) positions adjacent to the primary TMS electromagnet. This may help shape the resulting primary TMS field, as illustrated in FIG. 2B.

For example, FIG. 2B illustrates the resultant magnetic fields from the arrangement shown in FIG. 2A. In FIG. 2B, field 200 from coil 100 and field 210 from coil 110 have been narrowed (e.g., focused) when compared to the field shown in the unmodified case of FIG. 1B. In FIG. 2A the action of the focusing electromagnets, when activated to counterbalance the electromagnetic field of the primary TMS electromagnet results in “shaving off” the sides of the magnetic-field profiles 200 and 210 by indentations 220, 230, 240, and 250. The field representations shown in FIGS. 1B and 2B may be simplified version of the actual fields, however, the relative intensities may be reflective of the effects achieved.

As mentioned, any appropriate focusing electromagnet may be used. Generally, the focusing electromagnets are smaller than the primary electromagnet, so that the field emitted by the focusing electromagnet(s) effects only the peripheral region, but does not interfere with the peak field region and intensity of the primary TMS electromagnet. The focusing electromagnets are typically positioned so that their emitted field will counterbalance (and therefore reduce) the peripheral region of the primary focusing electromagnet. For example, the focusing electromagnets may be positioned in the peripheral regions of the primary electromagnet, away from the portion of the primary electromagnet that emits the highest intensity (the peak regions shown in FIG. 1B). In variations including primary electromagnets formed by a plurality of coils, as shown in FIG. 2A, the focusing electromagnets may be positioned adjacent to the region of the primary electromagnet that is lateral to the region where the coils meet.

In all of the examples provided herein, the focusing electromagnet is a powered electromagnet. In theory, a permanent magnet may also be used as a focusing magnet, however, in general, the power provided by an electromagnet is greater.

In operation, the focusing electromagnets are synchronously activated when the primary electromagnet is activated. The field emitted by the focusing electromagnets is configured to be oriented in the opposite orientation from that emitted by the primary electromagnet, so that the field from the smaller focusing electromagnets counterbalances a peripheral portion of the primary electromagnet's field, as indicated in FIG. 2B. In a preferred embodiment, the focusing magnets are smaller diameter than the primary magnet. Smaller electromagnets typically have turns of smaller diameter than larger electromagnets, and the magnetic field emitted from the smaller turns falls off more rapidly as a function of distance than does a field of the same strength (flux density) emitted by coils of larger diameter turns, as specified by the Biot-Savart Law. Accordingly, small magnets are suited to blocking portions of the magnetic field emitted by a larger primary coil without significantly impacting electrical current flow through the primary magnet.

Both the primary TMS electromagnet(s) and any focusing electromagnets may be powered by any appropriate power supply. For example, commercially available power sources may be used, or modified for use. In some variations, a commercially available power sources such as the Magstim Rapid² (Magstim Ltd., Wales, UK), which provides for pulsed magnetic fields, may be used.

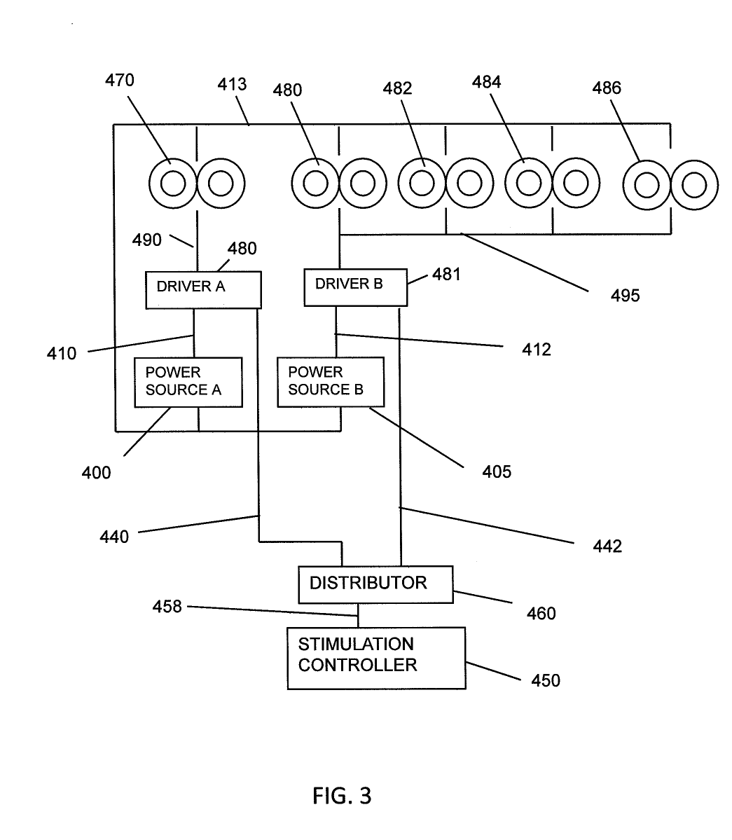

FIG. 3 illustrates one variation of a system for controller the powering of both a primary TMS electromagnet and a plurality of focusing electromagnets. In this variation, the primary TMS electromagnet whose magnetic field is to be focused (for example the double coil electromagnet made up of 100 and 110 in FIG. 2A) is powered by power source “A” 400 and the focusing electromagnets are powered by power source “B” 405. In some variations, the power sources powering the focusing electromagnets may be the same as the power source for the primary TMS electromagnet (the output of which may be inverted or otherwise modified for application to the focusing electromagnets). In other variations, multiple power sources may be used to power different focusing electromagnets. All of the electromagnets may share a common ground. For example, the common side of all the electromagnets in this example is connection 413. The primary TMS electromagnet 470 is also connected to the output of Driver “A” 480 via connection 490. The focusing electromagnets 480, 482, 484, and 486 are also connected to the output of Driver “B” 481, through connection 495. The power to be transmitted to electromagnet 470 via Driver “A” 480 is provided from Power Source “A” 400 via connection 410. The power to be transmitted to electromagnets 480, 482, 484, and 486 through Driver “B” 481 is provided from Power Source “B” 405 via connection 412. Distributor 460 provides the control for firing Driver “A” 480 and Driver “B” 481 under the control of Stimulation Controller 450 via connections 440 and 442, respectively with connection 458 providing the interface between Simulation Controller 450 and Distributor 460. In general, the distributor may coordinate the firing (e.g., powering) of the primary TMS electromagnet and the focusing electromagnet(s). In particular, the distributor may synchronize the firing of the one or more TMS electromagnets and the focusing electromagnets associated with a particular primary TMS electromagnet.

As indicated by the schematic, the activation of the focusing electromagnets is coordinated with the activation of the primary TMS electromagnet. In this variation, the stimulation controller (or “controller”) coordinates the activation so that the fields of the focusing electromagnets counter the field emitted by the primary TMS electromagnet during stimulation. In this variation the focusing electromagnets area shown schematically as double-coil electromagnets, but single-coil and other variations of electromagnets may be used, as mentioned above.

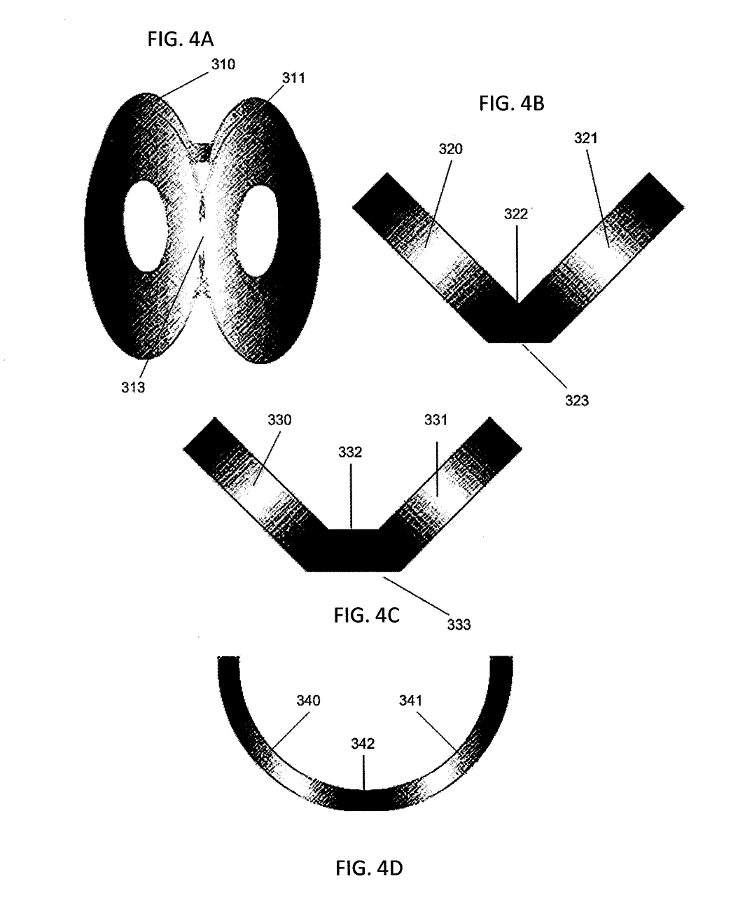

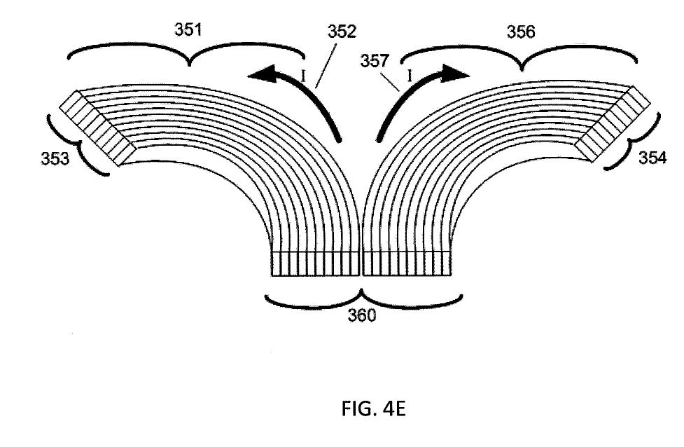

Another way to focus the emitted field of a primary TMS electromagnet which may be used in addition to the use of focusing magnets includes arranging the primary electromagnet into a focusing shape, as indicated by FIGS. 4A-4D. In general, a focusing shape is a shape having a planar region configured for placement nearest the target region (e.g., near the subject's head) and lateral margins that extend away from the planar region. The planar region typically corresponds to the region of the electromagnet from which the peak (e.g., the center) of the field would be emitted. The focusing shape may therefore exaggerate the peak region and attenuate the lateral marginal regions.

FIGS. 4A-4D illustrate alternative embodiments of coil shapes that are configured into focusing shapes capable of producing a relative focus in a magnetic field. In principle, by extending the lateral margins of the coil backwards (away from the patient's brain or body when in use) while keeping the central point of maximum intensity forward (and as close as possible to the intended target), that the central point is able to exert its effect on the target with less surrounding signal delivered to surrounding areas of a patient's brain or body.

In FIGS. 4A through 4D, the exterior insulating shell of a double coil, and the electrically conductive portions beneath, are intended to be implied by the shape of the overlying shell illustrated. FIG. 4E illustrates a cross-section of the conductive portions beneath the shell, with particular attention to the planar region of the central portion.

Double coils are used in these examples, but the principle of the focusing shape applies to single, quadruple, etc. coils of essentially any shape. In any of these examples, the electromagnets may be configured to include a planar region corresponding to the region of peak intensity of the emitted field and the regions peripheral to this may extend backwards, away from the subject when the electromagnet is to be used to stimulate a subject during TMS.

FIG. 4A shows a frontal view of a double coil TMS electromagnet consisting of coil 310 and coil 311, joined with a crossover at a central point 313, which forms an angle that places point 313 closer to the patient than the opposite margins of coils 310 and 311. Electrically, this coil may be similar to a standard double TMS coil, such as those manufactured by Magstim LTD (Wales, UK), or the Dantec (a subsidiary of Medtronic, Copenhagen, Denmark). Although coils having non-flat configurations (for example “butterfly” double coils) are commercially available from vendors including Magstim and Dantec, such magnets are oriented opposite those described herein, and do not include the important central planar region. While the reverse-face of these coils may be usable to exclude lateral field from the patient's head, the central portion of the field is weakened by the angular separation of the central coil wire elements making such coils weak in the reverse-faced position. In the magnet focusing shapes described herein, an important feature is that despite the bending of the lateral sections of the coil, the central section 260 is substantially planar, thereby maximizing the magnetic summation of the two adjacent sub-coils 351 and 356. By contrast, in commercially available coils each subcoil half of the double coil is wound upon a single plane, and the two flat component subcoils are simply set at angles to one another.

The planar region is configured so that it may be the region of the primary TMS electromagnet that is closest to the subject. The shape and size of the planar region may also be configured to help shape the emitted electromagnetic field. For example, the shape of the field may be round, oval, rectangular, square, semicircular, or the like. Elongated shapes (e.g., rectangular, square, or the like) may be particularly beneficial, though asymmetric face shapes may also be used. The planar region may be greater than 1 mm², greater than 2 mm², greater than 5 mm², greater than 10 mm², greater than 20 mm², etc. For example, in one variation the planar region of the coil turns are formed by the parallel turns in the central region of a 70 mm double coil, and the planar region measures approximately 4 centimeters across (and thus may have a cross-sectional area of approximately 10 to 20 cm²).

In FIG. 4A, the light colored areas of the drawing are intended to represent light reflections from the nearer portions of the apparatus shown, relative to the viewer. FIG. 4B shows a top-down view of a double coil like that shown in 4A. The double coil in 4B includes coils 320 and 321, with light colored areas again representing nearer portions of the apparatus from the perspective of the viewer. The medial portions of these coils, including the crossover are contained within face 323, which has a posterior angle 322. Face 323 is the surface that is placed, for example, on the scalp of a TMS patient, while the lateral margins of coils 320 and 321 face away from the patient's scalp. In an alternative embodiment, shown in FIG. 4C, coils 330 and 331 may be bent closer to their center, such that coil face 333 is wider than that shown in the previous figure, and the posterior angle becomes a posterior face 332. In yet another embodiment, shown in FIG. 4D, the sharp angles of the previous embodiments may be rounded so as to form a continuous curve. Here, coils 340 and 341 have a junction at point 343, but the angle at that point is continuous with the coils.

FIG. 4E illustrates a tri-planar cross-section of the conductive portions beneath the shell of the previously described FIGS. 4A through 4D, with particular attention to the planar configuration of the central portion. In particular, this coil is composed of concentric sub-coil 351 with lateral cross-section 353 and concentric sub-coil 356 with lateral cross-section 354. Electrical current I runs in direction 352 in subcoil 351 and in direction 357 in sub-coil 356. The lateral margins of each sub-coil (as seen at lateral cross-sections 353 and 354 are bent out of plane from the central portion of the coil where the two subcoils are adjacent, at section 360. Despite the bending of the lateral sections of the coil, central section 260 is substantially planar, thereby maximizing the magnetic summation of the two adjacent sub-coils 351 and 356.

In some variations, a TMS electromagnet may include both a focusing shape and one or more focusing electromagnets. For example, a primary TMS electromagnet may be configured so that the front of the electromagnet (the region configured to be closest to the subject's brain) includes a planar region corresponding to the peak of the emitted field. The rest of the primary TMS electromagnet may extend behind this planar region (e.g., the lateral margin regions around the planar region). Any of the devices or system described herein may include a frame or other structure to which the primary TMS electromagnet (or magnets) is mounted so that the system is configured for the planar regions to be closest to the subject's head. The primary TMS electromagnet may also include one or more focusing electromagnets positioned adjacent to the primary electromagnets (e.g., on the back or sides of the primary electromagnet). In particular, the focusing electromagnets may be adjacent to the lateral margin regions of the primary electromagnet, so that they may counterbalance these regions, thereby focusing the emitted field.

One application of such focused magnetic fields is to confine the magnetic stimulation to a narrower trajectory/narrower for more effectively targeting of neural tissue superficially or at depth. The same focusing principles described herein apply to constant as well as pulsed magnetic fields.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention, which is set forth in the following claims.

### REFERENCES

Avery, D. H., Holtzheimer III, P. E., Fawaz, W., Russo, Joan, Neumaier, J. and Dunner, D. L., Haynor, D. R., Claypoole, K. H., Wajdik, C. and P. Roy-Byrne, “A Controlled Study of Repetitive Transcranial Magnetic Stimulation in Medication-Resistant Major Depression,” Biological Psychiatry, 2005; 59:187-194.
Schneider, M. B. and D. J. Mishelevich, U.S. patent application Ser. No. 10/821,807 “Robotic apparatus for targeting and producing deep, focused transcranial magnetic stimulation” Magstim Web site: http://www.magstim.com/magneticstimulators/magstimacc/12494.html

What is claimed is:

1. A Transcranial Magnetic Stimulation system for stimulating a neuronal target, the system comprising:
at least one primary Transcranial Magnetic Stimulation electromagnet;
a focusing electromagnet adjacent to the primary Transcranial Magnetic Stimulation electromagnet; and
a stimulation controller configured to coordinate activation of the primary Transcranial Magnetic Stimulation electromagnet and the focusing electromagnet, so that the field emitted by the focusing electromagnet focuses the magnetic field emitted by the primary electromagnet.

2. The system of claim 1, further comprising a plurality of focusing electromagnets adjacent to the primary Transcranial Magnetic Stimulation electromagnet.

3. The system of claim 1, further comprising a frame to which the primary Transcranial Magnetic Stimulation electromagnet is supported.

4. The system of claim 1, further comprising a plurality of Transcranial Magnetic Stimulation electromagnets, wherein each of the Transcranial Magnetic Stimulation electromagnets is focused by one or more adjacent focusing electromagnets.

5. The system of claim 1, further comprising a distributor communicating with the stimulation controller.

6. The system of claim 1, further comprising a primary Transcranial Magnetic Stimulation power source and a focusing electromagnet power source.

7. A Transcranial Magnetic Stimulation system for stimulating a neuronal target, the system comprising:
at least one primary Transcranial Magnetic Stimulation electromagnet having a focusing shape, wherein the in a primary Transcranial Magnetic Stimulation electromagnet comprises a planar region corresponding to the area of maximal field intensity emitted by the primary Transcranial Magnetic Stimulation electromagnet, wherein the planar region is configured to be placed in the closest location to the intended target,
and wherein the more lateral regions of the primary Transcranial Magnetic Stimulation electromagnet are directed backwards and away from the planar region, whereby the magnetic field produced by lateral regions is separated from the neuronal target region.

8. The system of claim 7, wherein the primary Transcranial Magnetic Stimulation electromagnet comprises a double coil magnet.

9. The system of claim 7, wherein the primary Transcranial Magnet Stimulation electromagnet comprises a plurality of joined coils, and wherein the planar region is formed by the intersection of the coils, further wherein the coils are configured so that the current running through the coils in the intersection region travels in essentially the same direction for all of the coils.

10. A Transcranial Magnetic Stimulation system for stimulating a neuronal target, the system comprising:
at least one primary Transcranial Magnetic Stimulation electromagnet having a focusing shape;
a focusing electromagnet adjacent to the primary Transcranial Magnetic Stimulation electromagnet; and
a stimulation controller configured to coordinate activation of the primary Transcranial Magnetic Stimulation electromagnet and the focusing electromagnet so that the field emitted by the focusing electromagnet narrows and focuses the magnetic field emitted by the primary electromagnet.

11. A Transcranial Magnetic Stimulation method for stimulating a neuronal target tissue, the method comprising:
positioning at least one primary Transcranial Magnetic Stimulation electromagnet adjacent to a subject's head; and
emitting a magnetic field from the primary Transcranial Magnetic Stimulation electromagnet toward a neuronal target; and
focusing the field emitted by the Transcranial Magnetic Stimulation electromagnet by energizing a focusing electromagnet located adjacent to the primary Transcranial Magnetic Stimulation electromagnet, so that the field emitted by the focusing electromagnet counterbalances a portion of the magnetic field emitted by the primary electromagnet.

12. The method of claim 11, wherein the step of emitting a focused magnetic field comprises energizing a plurality of focusing electromagnets located adjacent to the primary Transcranial Magnetic Stimulation electromagnet.

13. The method of claim 11, wherein the step of emitting a focused magnetic field comprises controlling a distributor to synchronize the power supplied to the primary Transcranial Magnetic Stimulation Electromagnet and the focusing electromagnet so that they fire simultaneously.

14. The method of claim 11, further comprising aiming the primary Transcranial magnetic Stimulation electromagnet at a neuronal target.

15. A Transcranial Magnetic Stimulation method for stimulating a neuronal target tissue, the method comprising:
positioning at least one primary Transcranial Magnetic Stimulation electromagnet adjacent to a subject's head, wherein the primary Transcranial Magnetic Stimulation is configured in a focusing shape having a planar region corresponding to the area of maximal field intensity emitted by the primary Transcranial Magnetic Stimulation electromagnet and lateral regions extending backwards and away from the planar region;
orienting the primary Transcranial Magnetic Stimulation electromagnet so that the planar region is positioned toward the neuronal target; and
emitting a magnetic field from the primary Transcranial Magnetic Stimulation electromagnet toward the neuronal target.
